(19) United States
(12) Patent Application Publication
(10) Pub. No.: US 2025/0229022 A1
(43) Pub. Date: Jul. 17, 2025

(54) INJECTION DEVICE, INJECTION ASSEMBLY AND MONITORING DEVICE

(71) Applicant: STICHTING HET NEDERLANDS KANKER INSTITUUT-ANTONI VAN LEEUWENHOEK ZIEKENHUIS, Amsterdam (NL)

(72) Inventors: Christian Ulrich BLANK, Amsterdam (NL); Judith Marjan LIJNSVELT, Amsterdam (NL)

(21) Appl. No.: 18/684,964

(22) PCT Filed: Aug. 19, 2022
(86) PCT No.: PCT/NL2022/050476

§ 371 Date: Feb. 20, 2024

(30) Foreign Application Priority Data
Aug. 20, 2021 (NL) 2029005
Dec. 21, 2021 (NL) 2030202

(51) Int. Cl. A61M 5/172; A61B 5/00; A61B 5/145

(52) U.S. Cl. CPC A61M 5/1723; A61B 5/14514; A61B 5/14546; A61B 5/4266; A61B 5/746; A61M 2005/1726; A61M 2205/3303; A61M 2230/20

(57) ABSTRACT

A monitoring device for monitoring a hypocortiolism patient, the monitoring device comprising:—a needle for penetrating a skin of the patient, and—an analysis module fluidly connected with the needle, the analysis module comprising: o a first sensor, for measuring an inflammation level representative for the patient; and/or o a second sensor, for measuring a stress level representative for the patient; and wherein the monitoring device is configured to automatically trigger a notification in response to the first sensor measuring an inflammation level exceeding a first pre-defined threshold and/or in response to the second sensor measuring a stress level exceeding a second pre-defined threshold.

The present invention relates to an injection device. The present invention also relates to an injection assembly and a monitoring device.

Adrenal gland insufficiency, also known as hypocortisolism or Addison's disease, is a rare autoimmune disease wherein the human body does not produce (enough of) the steroid hormones cortisol and/or aldosterone. Approximately 100-140 per 1 million people suffer from this disease. In a secondary form of this disease control of the hypophysis does not function optimally. This also leads to a shortage of cortisol and/or aldosterone production by the human body. Another 150-280 per 1 million people suffer from this secondary form.

In cancer treatments wherein checkpoint inhibitor therapy is used, up to about 4% of the patients, and according to some neoadjuvant studies even a larger percentage of up to 13% of the patients develop hypocortisolism as a side effect of the cancer treatment. Thus, although the life of these patients may be saved as a result of a successful cancer treatment, their lives are endangered again (albeit to a lesser degree) by the development of hypocortisolism as a side-effect of the cancer treatment. These patients have not been accounted for in the above numbers of 100-140 and 150-180 per 1 million persons. As more and more patients are successfully treated against cancer with the use of checkpoint inhibitor therapy, the patient group suffering from hypocortisolism is increasing slowly but steadily. It is expected that the patient group suffering from hypocortisolism, in the Western world alone, will increase at a rate of at least 4,000 patients a year and up to or exceeding 16,000 patients a year. This disease has to be lived with for the entire life of the patient, as currently no cure has been found.

Patients with hypocortisolism have a shorter than normal life expectancy time. As long as medication is compliantly taken on a daily basis, it can be lived with for decades. The standard medication prescribed to patients suffering from hypocortisolism is the intake of hydrocortisone, an artificial steroid, several times a day. A typical dosage is 10 mg of hydrocortisone just after the patient awakes, and two additional dosages of 5 mg each during the remainder of the day. So, patients suffering from hypocortisolism are forced to swallow pills for the rest of their lives, which is unpleasant.

When insufficient dosages of hydrocortisone are taken, patients may develop a life-threating Addison crisis. This Addison crisis can already occur when a single dosage is forgotten and even more frequently occurs if, e.g. during times of stress or infections, an insufficient amount of an increased dosing is taken. An Addison crisis is a life-threatening situation. A standard cure against an Addison crisis is the injection of a large dosage of hydrocortisone. For that purpose, patients diagnosed with adrenal gland insufficiency have to carry emergency dosage with them at all times. Unfortunately, as the disease is quite rare, an Addison crisis is rarely recognized by bystanders, so although the cure is relatively simple and at hand, in many cases it is not administered.

This Addison crisis, as a result of under-administration of hydrocortisone, is so life-threatening, that patients are usually given more medication, than is strictly necessary to build up a buffer. However, recent studies show that this overmedication decreases the life expectancy of patients suffering from hydrocortisone deficiency.

When patients undertake stressful activities (such as an intervention at the dentist, motor-bike riding, taking an exam or attending events) or develop an infection, a higher dosage of hydrocortisone is needed. If a higher dosage is not taken some time before the activity is undertaken, the patient is at risk of developing the life-threatening Addison crisis, too. So, patients suffering from adrenal gland insufficiency need to plan their lives very carefully, be on the watch regarding any infections at all times and must not forget to take their medication. Undertaking spontaneous activities can, literally, be deadly.

So, although hypocortisolism can be lived with for a very long time all this makes that the disease strongly impairs the flexibility of the patients' lives in a negative way. Patients have to swallow pills for their entire life; cannot undertake spontaneous activities, have a shorter life expectancy and are always stressed about medication intake.

It is an object of the present invention to improve the safety and quality of life of such patients, to allow them to undertake events more spontaneously and make them less aware of having this disease. It is a further object of the present invention to increase the life expectancy of such patients.

Therefore, according to a first aspect of the present invention an injection device is presented for automatically administering a corticosteroid to a patient, the injection device comprising:
a fluid container for storing an injection fluid, the injection fluid comprising or containing the corticosteroid;
a needle for penetrating a skin of the patient, the needle being fluidly connected with the fluid container such that the injection fluid can be injected into the patient with the needle;
at least two of:
   a first sensor, for measuring an inflammation level representative for the patient;
   a second sensor, for measuring a stress level representative for the patient; and
   a third sensor, for measuring a corticoid level representative for the patient
      wherein the injection device is configured to automatically inject the injection fluid on more than one instance over the course of a 24 h period, according to a pre-defined basic dosage regime; and
wherein the injection device is configured to automatically inject an additional dosage of the injection fluid, on top of the basic dosage regime, in response to the first sensor measuring an inflammation level exceeding a first pre-defined threshold and/or in response to the second sensor measuring a stress level exceeding a second pre-defined threshold.

A second aspect of the present invention relates to an injection assembly for automatically administering a corticosteroid to a patient, the injection assembly comprising:
an injection member comprising:
a fluid container for storing an injection fluid, the injection fluid comprising or containing the corticosteroid;
a needle for penetrating a skin of the patient, the needle being fluidly connected with the fluid container such that the injection fluid can be injected into the patient with the needle; and
a sensor member comprising:
optionally, a needle for penetrating a skin of the patient, and
an analysis module, fluidly connected with the needle when a needle is present, the analysis module comprising at least two of:
   a first sensor, for measuring an inflammation level representative for the patient;
   a second sensor, for measuring a stress level representative for the patient; and
   a third sensor, for measuring a corticoid level representative for the patient;
wherein the injection member and the sensor member are communicatively coupled with each other, the injection member being configured to:
automatically inject the injection fluid on more than one instance over the course of a 24 h period, according to a pre-defined basic dosage regime; and
automatically inject an additional dosage of the injection fluid, on top of the basic dosage regime, in response to the first sensor measuring an inflammation level exceeding a first pre-defined threshold and/or in response to the second sensor measuring a stress level exceeding a second pre-defined threshold.

A third aspect of the present invention relates to a monitoring device for monitoring hypocortiolism patient, the monitoring device comprising:
optionally, a needle for penetrating a skin of the patient, and
an analysis module, fluidly connected with the needle when a needle is present, the analysis module comprising:
   a first sensor, for measuring an inflammation level representative for the patient; and/or
   a second sensor, for measuring a stress level representative for the patient; and
      wherein the monitoring device is configured to automatically trigger a notification in response to the first sensor measuring an inflammation level exceeding a first pre-defined threshold and/or in response to the second sensor measuring a stress level exceeding a second pre-defined threshold.

The first, second and third aspect of the present invention have in common that at least two sensors are present. The sensors determine a level of inflammation, and/or a level of stress and/or a level of corticoids representative for the patient. Depending on the height of these levels, either an additional dosage of an injection fluid is provided (when a needle is present) or a notification is triggered when the levels are too high (when it concerns stress and/or inflammation) or too low (when it concerns corticoid). This all happens automatically, without any involvement of the patient and preferably before the patient him- or herself notices that the levels are too high (or, when it concerns the corticoid level, before the levels are too low). This significantly increases the quality of life of the patients and, when seen over the entire patient group, will likely significantly reduce the number of Addison crises.

As will be described in more detail in the below, the level of inflammation, the level of stress and/or the level of corticoids can be obtained from a bodily fluid of the patient. Examples of suitable bodily fluids include but are not limited to blood, interstitial fluid, saliva and sweat. When the bodily fluid is obtained as blood or interstitial fluid, a needle is preferably present to ‘tap’ the bodily fluid from the inside of the body and transport it to the respective sensor. When the bodily fluid is obtained as saliva or sweat, the needle for obtaining a bodily fluid may be omitted and the bodily fluid may be ‘tapped’ in a non-invasive manner. The described bodily fluids all contain enough biomarkers to allow an analysis thereof with a sensor.

Advantageously for the first and the second aspect mainly, by automatically injecting a basic dosage regime with the injection device, the chance that a patient forgets a dosage is minimized. This leads to a significant increase in the safety for the patient and in quality of life for the patient as the stress and discomfort of taking medication several times a day is mitigated. Additionally, the constant stress and discomfort of not being able to forget medication is also resolved. This is expected to reduce the number of Addison crises, when looking at the patient group as a whole.

Further advantageously, for all aspects of the present invention, as the device is configured to inject an additional dosage of the injection fluid and/or to provide a notification when increased stress and/or inflammation levels are detected, the patient does not need to plan his/her life so carefully anymore. Instead, the patient can undertake events spontaneously without having to take medication a certain period of time before the activity is undertaken. An additional dosage will be provided automatically or the patient will be warned that an additional dosage should be taken. This also significantly increases the quality of life of the patient and reduce the number of Addison crises, when looking at the patient group as a whole.

Further advantageously, by monitoring the stress and/or inflammation level of the patient, the additional dosage may be administered already before any symptoms of an Addison crisis are showing. This reduces the stress conceived by patients as they nowadays have to watch these levels themselves, increasing the quality of life of the patients. Furthermore, this reduces the likelihood that an Addison crisis occurs.

Further advantageously, by monitoring the corticoid levels and by allowing for automatic injection of the injection fluid, the chronic overdosing can be avoided but may more closely resemble the natural corticoid production by “healthy” persons. This is expected to increase the life expectancy of the patients.

According to aspects of the present invention, a corticosteroid may be administered to a patient with an injection device or an injection member. As used herein, the injection device or injection member may e.g. be a personal device, worn by the patient at all times. For example, the injection device or member may be a wearable device or member, semi-permanently connected to a body of the patient (e.g. in the way a stoma is a wearable device). For example, the injection fluid injected by the injection device may be injected into the blood stream of the patient. In another example, the injection fluid may be injected into the interstitial fluid of the patient. As some patients are expected to dislike the permanent insertion of a needle in their skin, according to a third aspect of the present invention “only” the monitoring components are implemented, the monitoring member adapted to provide a notification when otherwise an additional dose would be injected. This notification then informs the patient that additional medication must be taken. For example, the notification can be an alarm, e.g. in the form of a sound signal, a vibratory signal, or a light signal. Alternatively and/or simultaneously, the notification can be a message or pop-up in an app of a mobile phone of a patient and/or relative of the patient, or on the device itself. Alternatively and/or simultaneously, the respective levels can be checked in real time on an app installed on a mobile device. For example, the notification may be activated on the monitoring device itself, and/or the monitoring device may e.g. communicate with a mobile device of the patient, a relative of the patient or a caretaker of the patient to trigger a notification there.

According to aspects of the present invention a corticosteroid, or any other equivalent or alternative medicament known to prevent a cortisol deficiency, is injected into the body of a patient. In principle, such a treatment with such a medicament is known; all medicaments known today and invented in the future may be injected with the present injection device or injection member.

According to aspects of the present invention, the corticosteroid is injected in a patient. For example, the patient is a human patient. However, the present injection device or member may also be suitable for non-human patients.

According to aspects of the present invention, the corticosteroid is automatically injected. As will be explained in the below, a standard dosage regime is automatically provided to the patient. When needed, on top of the standard dosage regime an additional dosage is provided to the patient, without the patient needing to monitor anything. The standard dosage regime is always provided, the additional dosage may be provided when the sensors provide a certain measurement signal.

According to aspects of the present invention, the injection device or assembly comprises a fluid container storing an injection fluid. Typically, the injection fluid will be an injection liquid, although this is not necessary per se. The fluid container is advantageously replaceable and may e.g. be a capsule, e.g. needing replacement every day, every week, every month, or at another interval. Preferably the replacement interval is longer than 24 hours to make the “hassle” for the patient to change the container at regular times as small as possible.

According to aspects of the present invention, the injection device or assembly further comprises a needle for penetrating the skin of the patient. Preferably the needle is permanently inserted in the skin, such that the injection fluid can be injected at all times when deemed necessary and/or according to the basic dosage regime. Alternatively, and equivalently the injection device or member may be attached to a (separate) needle that is permanently inserted in the skin of the patient. For example, the needle may mouth directly in a vein of the patient, or the needle may mouth in the interstitial fluid of the patient.

When the injection components and the monitoring components are integrated in the same device, one may refer to this single device as an injection device or an injection and monitoring device. Alternatively, there may be two devices: an injection member and, separately therefrom, a monitoring member. These two members together communicate with each other and may e.g. be worn on two different positions on the body. They may then be referred to as an injection assembly or an injection and monitoring assembly.

When the functionality as described herein is implemented in an assembly, there may be two needles: one to extract a bodily fluid to perform an analysis on, and one to inject the injection fluid. Optionally, also when the functionality as described herein is implemented as an injection device it may comprise a further, second, needle, e.g. for extracting a bodily fluid from the patient, the extracted fluid e.g. being used to perform tests such as the measurement of one or more blood or ISF (interstitial fluid) levels, in particular stress level or inflammation level. For example, the bodily fluid may be blood or interstitial fluid. For example, the bodily fluid may be reinjected into the body of the patient after extraction and analysis, e.g. through the same needle as through which the injection fluid is injected (when the injection member and the sensor member are integrated in one device), through the same needle as through which the bodily fluid is extracted, or through a yet further needle. It is noted that the bodily fluid on which the sensor works may alternatively be sweat and/or saliva. In that case, the bodily fluid may be obtained non-invasively, i.e. without a needle penetrating a skin of the patient.

According to aspects of the present invention, a pre-defined basic dosage regime is administered by the injection device or member. When seen over the course of one day, i.e. a 24 h period, according to the pre-defined basic dosage regime at several instances the injection fluid is injected. For example, a trace amount of injection fluid may be injected continuously, or a dosage may be injected at pre-set times, e.g. three times a day. This ensures that a drug level is established and maintained, automatically, without the risk that a patient forgets medication intake.

In the below, several embodiments of the present invention will be described. It will be clear to one reading this text that the embodiments as described may relate to one or more of the first, the second and the third aspect of the invention as defined in the above.

According to embodiments of the present invention, a first sensor measures an inflammation level representative for the patient. For example, this measurement may be performed continuously or at regular intervals. For example, this measurement may be performed in real time, e.g. via the blood, the ISF, the sweat, or the saliva of the patient. The inflammation level measured by the first sensor may be compared to a patient-specific first threshold value. When it is determined by the device that the inflammation level exceeds the threshold value, the threshold being defined at a level exceeding the normal inflammation level, this is an indication that more corticosteroid is needed than the standard dosage regime, as otherwise the patient faces the risk of entering a life-threatening Addison crisis. Accordingly, and advantageously, without any involvement of the patient, and preferably before the patient realizes that the inflammation level is increased, an additional dosage of the injection fluid is administered by the injection device or member and/or a notification, e.g. an alarm, a warning signal, or a message in an app, e.g. through a pop-up, is triggered. It is noted that one skilled in the art is familiar with various types of sensors that can measure inflammation levels of a patient; the present invention is not limited to a particular kind of sensor. Monitoring the inflammation levels is mainly done to prevent the occurrence of Addison crises and to increase the quality of life of the patient by taking away the stress of needing to monitor inflammation levels him- or herself.

According to embodiments of the present invention, a second sensor measures a stress level representative for the patient. For example, this measurement may be performed continuously or at regular intervals. For example, this measurement may be performed in real time, e.g. via the blood or the ISF of the patient. The stress level measured by the second sensor may be compared to a patient-specific second threshold value. When it is determined by the device that the stress level exceeds the second threshold value, the second threshold being defined at a level exceeding the normal stress level, this is an indication that more corticosteroid is needed than the standard dosage regime, as otherwise the patient faces the risk of entering a life-threatening Addison crisis. Accordingly, and advantageously, without any involvement of the patient, and preferably before the patient realizes that the stress level is increased, an additional dosage of the injection fluid is administered by the injection device or member and/or a warning signal or alarm is triggered. It is noted that one skilled in the art is familiar with various types of sensors that can measure stress levels of a patient; the present invention is not limited to a particular kind of sensor. Monitoring the stress levels is mainly done to prevent the occurrence of Addison crises and to increase the quality of life of the patient by taking away the stress of needing to monitor inflammation levels him- or herself.

In an embodiment of the present invention a third sensor measures a corticoid level, in particular cortisol and/or 11-deoxycortisol in the body of the patient. For example, the third sensor may measure a level of a naturally produced corticoid. Alternatively and/or simultaneously, the third sensor may measure a level of a man-made corticoid, also known as steroid. Preferably, any such third sensor is communicatively coupled to a controller. When the controller, based on the measurements of the third sensor, notes that despite the administration of the normal dosage regime the corticoid level in the body of the patient drops, it may increase said normal dosage regime, or perform other actions such as administering an additional dosage or trigger the alarm. As such, the third sensor may work as a “fail-safe” option to still inject an additional dosage or trigger the notification when the other two sensors have apparently failed. For example, this measurement by the third sensor may be performed continuously or at regular intervals. For example, this measurement may be performed in real time, e.g. via the blood or the ISF of the patient. The level measured by the third sensor may be compared to a patient-specific third threshold value. When it is determined by the device that the corticoid level drops below the third threshold value, the threshold being defined at a level below the normal corticoid level, this is an indication that more corticosteroid may be needed, as otherwise the patient faces the risk of entering a life-threatening Addison crisis. Accordingly, and advantageously, without any involvement of the patient, and preferably before the patient realizes that the corticoid level is decreased, an additional dosage of the injection fluid may be administered by the injection device or member and/or the alarm may be triggered. It is noted that one skilled in the art is familiar with various types of sensors that can measure corticoid levels of a patient; the present invention is not limited to a particular kind of sensor. Advantageously, by using a corticoid sensor the corticoid level in the blood or ISF or other bodily fluid can be monitored continuously, to determine the trend of said level and to correct early on when a deviation from the normally expected pattern is noted. Providing a corticoid sensor in combination with an injection needle for injecting the injection fluid continuously, or regularly in small dosages each time, is mainly advantages to increase the life expectancy of the patient, as in this way a chronic over-medication can be prevented and, rather, the normal production of corticoid can be followed by the administering of small dosages continuously or frequently. It is also expected that the quality of life of patients is increased when small dosages are administered frequently compared to relatively large dosages infrequently.

It is noted that, for all three sensors, the bodily fluid on which the measurements are performed may be obtained invasively, i.e. with a needle, e.g. when the bodily fluid is blood or ISF. Alternatively and/or additionally, the bodily fluid on which the measurements are performed may be obtained noninvasively, e.g. without a needle, e.g. when the bodily fluid is sweat or saliva.

Preferably, the injection device is further configured to inject an additional dosage of the injection fluid when each of the stress level and the inflammation level exceed their normal values by a pre-defined amount or percentage, without the first or the second threshold value being reached individually. Of course, in such an embodiment the first and the second sensors would have to be present.

In an embodiment of the present invention, the injection device or injection member further comprises a controller that is communicatively coupled to at least two of the first sensor, the second sensor and the third sensor, as well as an actuator for forcing the injection fluid from the fluid container into the needle, the actuator being communicatively coupled to the controller. In particular, based on the information obtained by the sensors and a comparison of the levels measured by said sensors against the threshold values, the controller can actuate the actuator to administer the additional dosage when deemed necessary. Advantageously, all this can occur without any active involvement of the patient.

In an embodiment of the present invention, at least one dosage of the pre-defined basic dosage regime is injected at an instance corresponding to 0-180 minutes before the patient is set to awake. The time at which the patient is set to awake may differ between different patients and may even differ between different days of the week for a certain patient. For example, the injection device may be synchronised with a mobile phone or other alarm of the patient, e.g. via an app, the injection device logging the time at which the alarm of the patient goes off, and administering a dosage in the interval of 0-180 minutes before that time. Advantageously, it is expected by the applicant that compared to an intake of medication just after the patient wakes up, automatically administering the medication already before the patient wakes up will help to make the patient feel better, and will further improve the quality of life of the patient.

In an embodiment of the present invention, the corticosteroid is a glucosteroid, in particular hydrocortisone, prednisone, prednisolone, or fludrocortisone. These are examples of known medications. However, it is repeated that the present inventive concept does not lie in the type of medication given, but the how and/or when the medication is given, as described in the above.

In an embodiment of the present invention, the basic dosage regime provides an active dosage of 10 mg hydrocortisone at one instance over the course of the 24 h period and two active dosages of 5 mg hydrocortisone at two different instances over the course of the 24 h period. Preferably the highest dosage is given in the morning, e.g. before the patient is set to awake, and the other two dosages are given over the course of the day.

In an alternative embodiment of the present invention the basic dosage regime provides a dosage regime in a pattern that corresponds to a natural cortisol production regime in patients without hypocortiolism. It is expected that such a dosage regime has a lower “overmedication” and as a result increases the life expectancy of the patient.

In an embodiment of the present invention, the volume of the additional dosage corresponds to 1-10× the basic daily intake according to the pre-defined basic dosage regime. Advantageously, some of the presently-known corticosteroids used in the treatment of hypocortisolism can in principle be given in relatively high dosages on an exceptional basis without being directly harmful. It is a shortage of the medication which is life-threatening, within normal boundaries it is virtually impossible to administer too much when an additional dosage is administered. Therefore, to take away the threat of under-medication when the inflammation level and/or the stress level increases, preferably the relatively high additional dosage of 1-10× the basic daily intake is injected at once.

In an embodiment of the present invention, the first sensor measures the inflammation level by monitoring the c-reactive protein, CRP, level in the body of the patient and/or the interleukine-6, IL-6, level in the body of the patient. These indicators are just two examples of indicators known to correlate with the inflammation level in the body and for which suitable sensors presently exist; in alternative embodiments the inflammation level may be tracked through the measurement of other indicators with suitable sensors. It is not necessary, but it is possible, that the inflammation level is measured by inserting e.g. a needle in the bod of the patient (e.g. through the skin). In one example, the measurement may be performed on biomarkers present in the blood of the patient, the blood e.g. obtained via a needle in an invasive manner. In another example, the measurement may be performed on biomarkers present in the ISF of the patient, the ISF e.g. obtained via a needle in an invasive manner. In yet another example, the measurement may be performed on biomarkers present in the sweat of the patient, the sweat e.g. obtained from the skin of the patient in a non-invasive manner. In a yet further example, the measurement may be performed on biomarkers present in the saliva of the patient, the saliva e.g. obtained from the oral cavity of the patient in a non-invasive manner. In an embodiment of the present invention, the second sensor measure the stress level by monitoring the adrenaline level in the body of the patient and/or the noradrenaline level in the body of the patient. These indicators are just two examples of indicators known to correlate with the stress level in the body and for which suitable sensors presently exits; in alternative embodiments the stress level may be tracked through the measurement of other indicators with suitable sensors. It is noted that (nor) adrenaline is known in the U.S.A. under the name (nor) epinefine; the two meaning the exact same thing. It is not necessary, but it is possible, that the stress level is measured by inserting e.g. a needle in the bod of the patient (e.g. through the skin). In one example, the measurement may be performed on biomarkers present in the blood of the patient, the blood e.g. obtained via a needle in an invasive manner. In another example, the measurement may be performed on biomarkers present in the ISF of the patient, the ISF e.g. obtained via a needle in an invasive manner. In yet another example, the measurement may be performed on biomarkers present in the sweat of the patient, the sweat e.g. obtained from the skin of the patient in a non-invasive manner. In a yet further example, the measurement may be performed on biomarkers present in the saliva of the patient, the saliva e.g. obtained from the oral cavity of the patient in a non-invasive manner. In alternative embodiments the stress level may be tracked through the measurement of other indicators with suitable sensors (e.g. hart rate, skin sweat).

In an embodiment of the present invention, the first and/or second pre-defined threshold for triggering the administration of the additional dosage corresponds to an increase of at least 50% of the respective, user-specific, normal inflammation and/or stress level. The natural, baseline inflammation and/or stress level may not be exactly the same for different patients and may also vary during the day, so preferably this baseline level is first determined on a patient-specific basis before the threshold is defined. For example, the threshold may be set at a level of 50% above the baseline level of a patient. In other embodiments, the threshold may be set at other levels above the baseline level.

In an embodiment of the present invention the injection device or member is wearable on the skin of the patient, e.g. on the arm, the leg, the abdomen, or another place, preferably a place where it can be hidden under clothes worn by the patient. When the patient is a human, this advantageously hides the injection device or member from sight for other persons.

In an embodiment of the present invention the inflammation level and/or the stress level and/or the corticoid level is obtained by bypassing blood of the patient across the first and/or the second and/or the third sensor. However, it may also be possible to obtain the respective level without passing blood across the first and/or the second and/or the third sensor depending on the exact type of sensor that is used. For example, the measurement may be performed on another bodily fluid, in particular interstitial fluid, or without bypassing any bodily fluid and in a non-invasive manner, in particular by performing a measurement “through the skin” or by performing the measurement on a bodily fluid, e.g. sweat or saliva, that may be obtained in a non-invasive manner.

In an embodiment of the present invention, the inflammation level and/or the stress level and/or the corticoid level is obtained by channeling sweat through microchannels of a patch, the patch applied on the skin of the patient, towards the first and/or the second and/or the third sensor.

These and other aspects of the present invention will now be elucidated further with reference to the attached figures. In these figures:

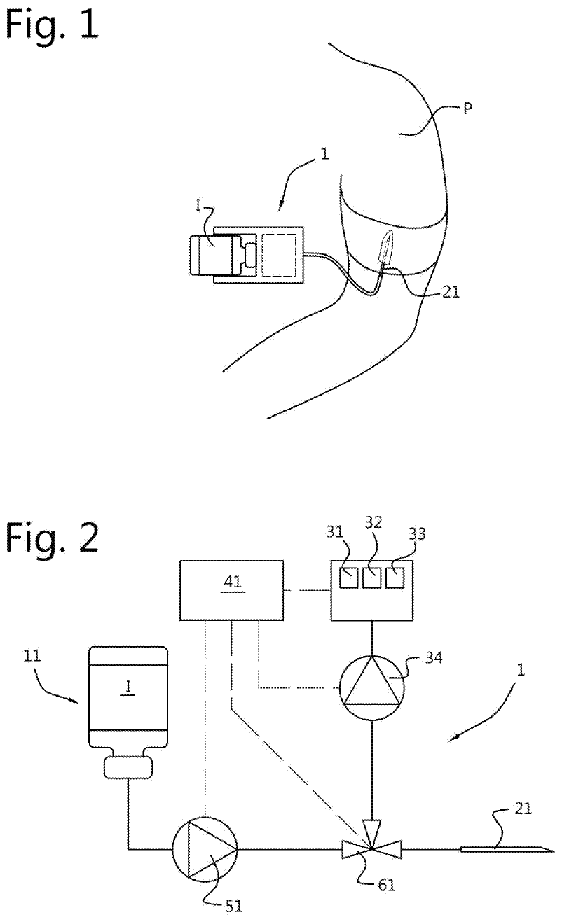

FIG. 1 schematically illustrates an embodiment of the injection device according to the present invention, with a needle of the injection device inserted into an arm of a patient;

FIG. 2 schematically illustrates different components the injection device of FIG. 1;

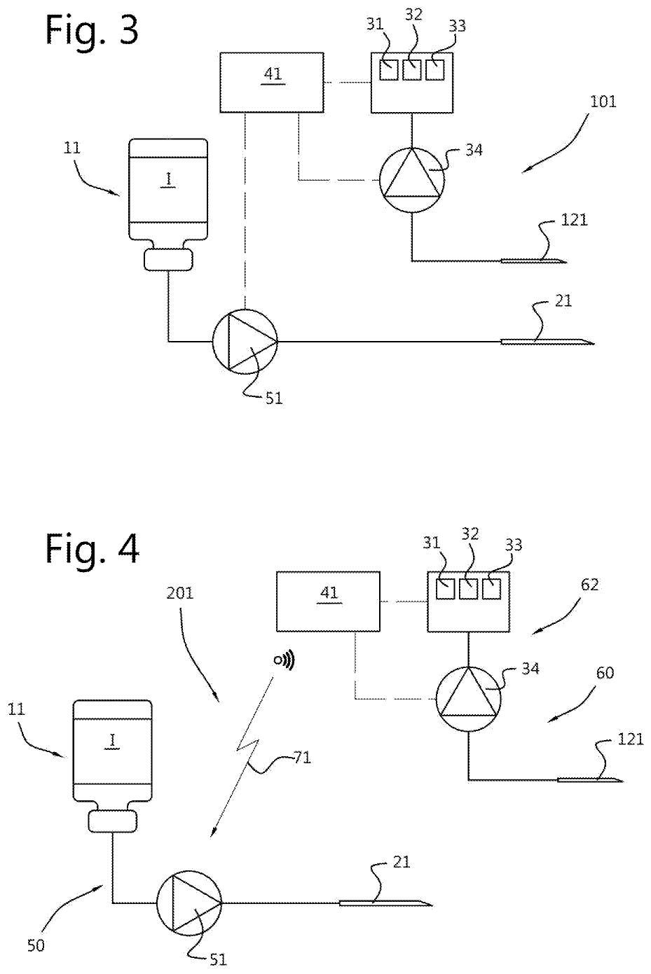

FIG. 3 schematically illustrates a further embodiment of the injection device according to the present invention;

FIG. 4 schematically illustrates an embodiment of an injection assembly according to the present invention;

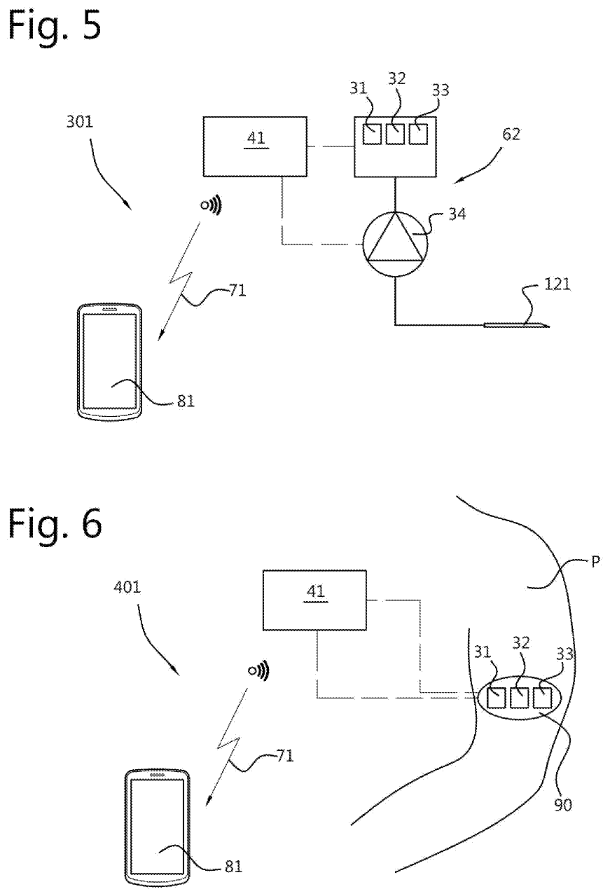

FIG. 5 schematically illustrates an embodiment of a monitoring device according to the present invention; and

FIG. 6 schematically illustrates a further embodiment of a monitoring device according to the present invention.

FIG. 1 schematically shows an arm of a patient P. In the shown example, the patient P is a human being, but in other examples the patient P may be non-human. In the shown example the injection device 1 has a needle 21, that is inserted into an arm of the patient P, the needle 21 penetrating the skin of the arm. However, in other examples the needle 21 may be inserted in other bodily parts of the patient, e.g. the leg, the abdomen, or any other place. Preferably the injection device 1 can be hidden from sight completely, e.g., by being wearable on the body of the patient P and/or by being wearable under clothes worn by the patient P.

In the shown example the injection device 1 comprises a needle 21. As one skilled in the art will understand, the device 1 may alternatively be connected to a catheter while applying the same inventive concept.

In the shown example the device 1 comprises a liquid container, e.g. embodied as an ampoule or another container, the container preferably being replaceable and storing the injection fluid I. The injection container and the needle 21 are fluidly connected, such that the injection fluid I can flow from the injection container into the needle 21. Through the needle 21 the injection fluid I is injected into the body of the patient P. For example, the injection fluid I is injected directly in the blood stream of the patient P, e.g. by injecting the injection fluid I in a vein.

The injection fluid I comprises or contains a corticosteroid, more in particular a glucosteroid, even more specifically the glucosteroid hydrocortisone or the glucosteroid fludrocortisone.

The injection fluid I is automatically administered into the body of the patient P by the injection device 1 according to a pre-defined basic dosage regime. For example, the pre-defined basic dosage regime may contain three injections over the course of a day. For example, a first injection may be administered once the patient has awoken. Alternatively, and advantageously, the first injection may be administered before the patient P has awoken, e.g. 0-180 minutes before the patient P is set to awake. For example, a second injection may be administered around noon, e.g. in between 11AM and 2 PM local time. For example, a third injection may be administered around diner time, e.g. between 4 PM and 11 PM local time. For example, the first injection may contain a higher dose of the active ingredient than the latter two injections. For example, when hydrocortisone is administered, the first injection may comprise around 10 mg of the active ingredient, and the later two injections may comprise around 5 mg of the active ingredient.

Turning to FIG. 2, some more components of the injection device 1 are shown. Again visible are needle 21 and fluid container 11 comprising the injection fluid I. These have been described in the above and will not be elaborated upon here. Indicated with reference numerals 31, 32 and 33 are first, second and third sensors.

The first sensor 31 is configured for measuring an inflammation level representative for the patient. This may e.g. be done by monitoring the c-reactive protein, CRP, level in the body of the patient. Alternatively, and/or simultaneously this may be done by monitoring the interleukine-6, IL-6 level in the body of the patient. Monitoring of the CRP and/or IL-6 level may e.g. be done by bypassing some blood of the patient through the needle 21, guiding it along the sensor 31, and determining the CRP and/or IL-6 level from the bypassed blood. After the analysis is performed, the bypassed blood may be re-injected into the body of the patient through needle 21 again.

The second sensor 32 is configured for measuring a stress level representative for the patient. This may e.g. be done by monitoring the adrenaline level in the body of the patient. Alternatively, and/or simultaneously this may be done by monitoring the noradrenaline level in the body of the patient. Monitoring of the adrenaline and/or noradrenaline level may e.g. be done by bypassing some blood of the patient P through the needle 21, guiding it along the sensor 32, and determining the adrenaline and/or noradrenaline level from the bypassed blood. After the analysis is performed, the bypassed blood may be re-injected into the body of the patient through needle 21 again.

The third sensor 33 is configured for measuring a corticoid level representative for the patient. Monitoring of the corticoid level may e.g. be done by bypassing some blood of the patient through the needle 21, guiding it along the sensor 33, and determining the corticoid level from the bypassed blood. After the analysis is performed, the bypassed blood may be re-injected into the body of the patient through needle 21 again.

For example, blood may bypass from the body of the patient by operating pump 34 and opening 3-way valve 61 in such a way that blood may flow from the needle 21 towards the respective sensor(s) 31, 32, 33.

For example, blood may be returned into the body of the patient by operating three-way valve 61 in such a way that blood may flow back from the respective sensor(s) 31, 32, 33 into the needle 21. For example, pump 34 may then be operated in the direction opposite to the direction it is operated in when blood is extracted from the body of the patient.

The measurements obtained by the sensors 31, 32, 33 may be sent to a controller 41. The controller 41 may be configured for comparing the inflammation levels, the stress levels and/or the corticoid levels measured by the sensors 31, 32, 33 to respective pre-defined threshold levels stored in the controller 41. Once the controller 41 determines that at least one of the inflammation level, the stress level and/or the corticoid level exceeds a respective threshold, e.g. by surpassing a certain value or by dipping below a certain value, the controller 41 may instruct valve 61 to open and pump 51 to activate and to inject an additional dosage of the injection fluid I, on top of the basic dosage regime, into the patient. All these actions are performed automatically, without involvement of the patient.

For example, the additional dosage injected may correspond to 1-10 times the basic daily intake according to the basic dosage regime.

For example, the first and/or second pre-defined threshold for triggering the administration of the additional dosage may relate to an increase of at least 50% of the respective, patient-specific, baseline inflammation and/or stress level.

For example, the third threshold for triggering the administration of the additional dosage may relate to a decrease of at least 20% below a respective, patient-specific, baseline corticoid level.

FIG. 3, compared to FIG. 2, shows an adapted version of injection device 101 with a second needle 121. The first needle 21 in this version is for injection of an injection fluid I only; the second needle 121 is for the removal and re-injection of a bodily fluid to perform tests on with the sensors 31, 32, 33. For example first needle 21 may be inserted into a blood vessel of the patient, wherein the smaller second needle 121 is inserted into the skin of the patient and extracts ISF.

In alternative embodiments the second needle 121 may be replaced by a patch, such as a patch that is schematically shown in FIG. 6. For example, the patch can be applied on the skin (instead of in the skin) to e.g. obtain sweat of the patient, the tests with sensors 31, 32, 33 being performed on the sweat.

FIG. 4, compared to FIGS. 2 and 3, shows an injection assembly 101. In the embodiment shown in FIG. 4 the injection member 50 comprising fluid container 11 and needle 21 is physically detached from sensor member 60 comprising second needle 121 and analysis module 62. The injection member 50 and the sensor member 60 can for example be worn on two different locations of a body of a patient. The injection member 50 and the sensor member 50 are communicatively coupled 71 to each other, e.g. via a 4G, 5G, Bluetooth, or wired connection. As described for FIG. 3, in the embodiment of FIG. 4 the needle 121, to be inserted into the skin of the patient, may be replaced by a patch to be applied on the skin of the patient.

FIG. 5, compared to the other figures, shows an embodiment of a monitoring device 301 without injection needle, especially adapted to those patients that do not wish to have an injection needle inserted in a blood vessel all the time. It comprises essentially only the sensor member of the injection assembly 101 shown in FIG. 4.

FIG. 6, compared to FIG. 5, shows an embodiment of a monitoring device 401 without any needle, especially adapted to those patients that fear needles. It comprises a patch 90 that can be applied on the skin of the patient and on which sensors 31, 32, 33 are arranged that can monitor the sweat for the relevant biomarkers as described herein.

### LIST OF REFERENCE NUMERALS

1 injection device
11 fluid container
21 first needle
121 second needle
31 first sensor
32 second sensor
33 third sensor
34 valve
41 controller
50 injection member
51 actuator
60 sensor member
61 three-way valve
62 analysis module
71 communication
81 mobile device
90 patch
101 injection device
201 injection assembly
301 monitoring device
401 monitoring device
injection fluid
P patient

1. A monitoring device (301) for monitoring a hypocortiolism patient, the monitoring device comprising:
a needle (121) for penetrating a skin of the patient (P), and
an analysis module (62) fluidly connected with the needle (61), the analysis module (62) comprising:
a first sensor (31), for measuring an inflammation level representative for the patient (P); and/or
a second sensor (32), for measuring a stress level representative for the patient (P); and
wherein the monitoring device (301) is configured to automatically trigger a notification in response to the first sensor (31) measuring an inflammation level exceeding a first pre-defined threshold and/or in response to the second sensor (32) measuring a stress level exceeding a second pre-defined threshold.

2. An injection device (1) for automatically administering a corticosteroid to a patient (P), the injection device (1) comprising:
a fluid container (11) for storing an injection fluid (I), the injection fluid (I) comprising or containing the corticosteroid;
a needle (21) for penetrating a skin of the patient (P), the needle (21) being fluidly connected with the fluid container (11) such that the injection fluid (I) can be injected into the patient (P) with the needle (21);
at least two of:
a first sensor (31), for measuring an inflammation level representative for the patient (P);
a second sensor (32), for measuring a stress level representative for the patient (P); and
a third sensor (33), for measuring a corticoid level representative for the patient (P);
wherein the injection device (1) is configured to:
automatically inject the injection fluid (I) on more than one instance over the course of a 24 h period, according to a pre-defined basic dosage regime; and
automatically inject an additional dosage of the injection fluid (I), on top of the basic dosage regime, in response to the first sensor (31) measuring an inflammation level exceeding a first pre-defined threshold and/or in response to the second sensor (32) measuring a stress level exceeding a second pre-defined threshold.

3. The injection device according to claim 2, further comprising a controller (41) that is communicatively coupled to the at least two of the first sensor (31), the second sensor (32) and the third sensor (33), and further comprising an actuator (51) for forcing the injection fluid (I) from the fluid container (11) into the needle (21), the actuator (51) being communicatively coupled to the controller (41).

4. The injection device according to claim 2, wherein at least one dosage of the pre-defined basic dosage regime is injected at an instance corresponding to 0-180 minutes before the patient (P) is set to awake.

5. The injection device according to claim 2, wherein the corticosteroid is in particular hydrocortisone, fludrocortisone, prednisone, or prednisolone.

6. The injection device according to claim 2, wherein the basic dosage regime provides an active dosage of 10 mg hydrocortisone at one instance over the course of the 24 h period and two active dosages of 5 mg hydrocortisone at two different instances over the course of the 24 h period.

7. The injection device according to claim 2, wherein the basic dosage regime provides a dosage regime in a pattern that corresponds to a natural cortisol production regime in patients without hypocortisolism.

8. The injection device according to claim 2, wherein the volume of the additional dosage corresponds to 1-10× the basic daily intake according to the pre-defined basic dosage regime.

9. The injection device according to claim 2, wherein the device comprises a first sensor (31), the first sensor (31) measuring the inflammation level by monitoring the c-reactive protein, CRP, level in the body of the patient (P) and/or the interleukine-6, IL-6, level in the body of the patient (P).

10. The injection device according to claim 2, wherein the device comprises a second sensor (32), the second sensor (32) measuring the stress level by monitoring the adrenaline level in the body of the patient (P) and/or the noradrenaline level in the body of the patient (P).

11. The injection device according to claim 2, wherein the device comprises the third sensor (33), the third sensor (33) for measuring the corticoid level by monitoring the 11-deoxycortisol level in the body of the patient (P) and/or the cortisol level in the body of the patient (P).

12. The injection device according to claim 2, wherein the first and/or second pre-defined threshold for triggering the administration of the additional dosage corresponds to an increase of at least 50% of a respective, patient-specific, baseline inflammation and/or stress level.

13. The injection device according to claim 2, wherein the injection device (1) is wearable on the skin of the patient (P), e.g. on the arm, the leg, the abdomen, or another place, preferably a place where it can be hidden under clothes worn by the patient (P).

14. The injection device according to claim 2, wherein the inflammation level and/or the stress level and/or the corticoid level is obtained by bypassing interstitial fluid of the patient (P) through the injection device (1) and across the first (31) and/or second (32) and/or third sensor (33).

15. The injection device according to claim 2, wherein the inflammation level and/or the stress level and/or the corticoid level is obtained by bypassing blood of the patient (P) through the injection device (1) and across the first (31) and/or second (32) and/or third sensor (33).

16. The injection device according to claim 2, wherein the device comprises a patch configured to be worn on a skin of the patient, the patch comprising flow channels, in particular microchannels, for transporting sweat of the patient towards the first (31) and/or second (32) and/or third sensor (33), and wherein the inflammation level and/or the stress level and/or the corticoid level is obtained from said sweat.

17. An injection assembly (201) for automatically administering a corticosteroid to a patient (P), the injection assembly (201) comprising:
an injection member (50) comprising:
a fluid container (11) for storing an injection fluid (I), the injection fluid (I) comprising or containing the corticosteroid;
a needle (21) for penetrating a skin of the patient (P), the needle (21) being fluidly connected with the fluid container (11) such that the injection fluid (I) can be injected into the patient (P) with the needle (21); and
a sensor member (60) comprising:
a needle (121) for penetrating a skin of the patient (P), and
an analysis module (62) fluidly connected with the needle (61), the analysis module comprising at least two of:
a first sensor (31), for measuring an inflammation level representative for the patient (P);
a second sensor (32), for measuring a stress level representative for the patient (P); and
a third sensor (33), for measuring a corticoid level representative for the patient (P);
wherein the injection member (50) and the sensor member (60) are communicatively coupled (71) with each other, the injection member (50) being configured to:
automatically inject the injection fluid (I) on more than one instance over the course of a 24 h period, according to a pre-defined basic dosage regime; and
automatically inject an additional dosage of the injection fluid (I), on top of the basic dosage regime, in response to the first sensor (31) measuring an inflammation level exceeding a first pre-defined threshold and/or in response to the second sensor (32) measuring a stress level exceeding a second pre-defined threshold.
